(19) United States
(12) Patent Application Publication
(10) Pub. No.: US 2023/0230248 A1
(43) Pub. Date: Jul. 20, 2023

(54) INFORMATION PROCESSING APPARATUS, INFORMATION DISPLAY APPARATUS, INFORMATION PROCESSING METHOD, INFORMATION PROCESSING SYSTEM, AND STORAGE MEDIUM

(71) Applicant: CANON KABUSHIKI KAISHA, Tokyo (JP)

(72) Inventor: Satoko Ohno, Kanagawa (JP)

(21) Appl. No.: 18/191,643

(22) Filed: Mar. 28, 2023

Related U.S. Application Data
(63) Continuation of application No. PCT/JP2021/038598, filed on Oct. 19, 2021.

(30) Foreign Application Priority Data
Oct. 26, 2020 (JP) 2020-179043

(51) Int. Cl. G06T 7/00; G06T 7/62; G16H 20/00; G16H 30/20; G16H 50/20

(52) U.S. Cl. CPC G06T 7/0012; G06T 7/62; G16H 20/00; G16H 30/20; G16H 50/20; G06T 2207/20084; G06T 2207/30068; G06T 2207/30096

(57) ABSTRACT

An information processing apparatus includes an estimation unit configured to estimate a diagnosis result for a medical image of a subject by using a learning model learned by a set of a medical image and a diagnosis result of the medical image, and an output unit configured to output a candidate for a treatment method to be applied to the estimated diagnosis result and an evaluation value for the candidate for the treatment method.

### CROSS-REFERENCE TO RELATED APPLICATIONS

This application is a Continuation of International Patent Application No. PCT/JP2021/038598, filed Oct. 19, 2021, which claims the benefit of Japanese Patent Application No. 2020-179043, filed Oct. 26, 2020, both of which are hereby incorporated by reference herein in their entirety.

### TECHNICAL FIELD

The present disclosure relates to an information processing apparatus, an information display apparatus, an information processing method, an information processing system, and a storage medium.

### BACKGROUND ART

In recent years, the amount of medical image data to be interpreted has increased as imaging apparatuses have become more powerful and imaging has become more frequent, but there is a shortage of doctors.

One system that has been used to solve the above problems is a computer-aided diagnosis (CAD) system, which uses a computer to analyze medical images and present information to help a doctor interpret the images.

### CITATION LIST

### Patent Literature

PTL 1 Japanese Patent Laid-Open No. 2017-386

However, the number of doctors is still insufficient and the time available to diagnose each subject is limited. In addition, there is no way to resolve the subjects' desire to judge the appropriateness of the treatment presented by the doctor with reference to other information.

Therefore, it can therefore be difficult for a subject to determine whether the treatment suggested by a doctor as a result of diagnosis by imaging is an appropriate option among the treatments available.

### SUMMARY OF INVENTION

In view of the above problems, the present disclosure provides a method of presenting information that enables a user to determine the efficacy of a treatment method for a disease.

In addition, each embodiment of the present disclosure also provides a function and an effect which are not achieved by conventional techniques.

In an aspect, the present disclosure provides an information processing apparatus including an estimation unit configured to estimate a diagnosis result for a medical image of a subject by using a learning model learned by a set of a medical image and a diagnosis result of the medical image, and an output unit configured to output a candidate for a treatment method to be applied to the estimated diagnosis result and an evaluation value for the candidate for the treatment method.

According to the present disclosure, it is possible to present information that allows a user to determine the validity of a treatment method for a disease.

Further features of the present invention will become apparent from the following description of exemplary embodiments with reference to the attached drawings.

### BRIEF DESCRIPTION OF DRAWINGS

FIG. 1 is a diagram illustrating an example of an information processing system according to a first embodiment.

FIG. 2 is a diagram illustrating an example of a configuration of an information processing apparatus according to the first embodiment.

FIG. 3 is a flowchart illustrating an example of a process performed in the information processing system according to the first embodiment.

FIG. 4A is a diagram illustrating an example of a process of generating a learning model according to the first embodiment.

FIG. 4B is a diagram illustrating an example of a process of generating a learning model according to the first embodiment.

FIG. 5 is a diagram illustrating an example of a UI (user interface) of an information display apparatus according to the first embodiment.

FIG. 6 is a diagram illustrating an example of an information processing system according to a first modification.

### DESCRIPTION OF EMBODIMENTS

Preferred embodiments of the information processing apparatus according to the present disclosure are described in detail below with reference to the accompanying drawings. However, constituent elements described in the embodiments are merely examples, and the technical scope of the information processing apparatus according to the present disclosure is not limited by the embodiments described below, but is determined by the scope of the claims. In addition, the present disclosure is not limited to the following embodiments, and various modifications (including organic combinations of embodiments) are possible based on the gist of the disclosure, and they are not excluded from the scope of the disclosure. That is, configurations obtained by combining embodiments and modifications thereof described later are also included within the aspects of the present disclosure.

### First Embodiment

An information processing apparatus according to a first embodiment presents information that enables a user to determine the validity of a treatment method derived from diagnosis results of medical images obtained by various medical imaging apparatuses (modalities), such as computed tomography apparatuses (hereinafter referred to as CT apparatuses).

More specifically, for example, a CT image of the chest including the lesion of a subject is first obtained by a CT apparatus, and then a diagnosis result is estimated by a learning model using the obtained CT image as input data. Then, from the estimated diagnosis results, a highly similar previous diagnosis result is extracted, and a treatment method performed for the extracted diagnosis is identified. Information about the identified treatment method is then presented to a user.

Note that the medical imaging apparatus is not limited to the above, and may be an MRI apparatus, a three-dimensional ultrasonic imaging apparatus, a photoacoustic tomography apparatus, a PET/SPECT apparatus, an OCT apparatus, a digital radiography apparatus, or the like. The area to be imaged is not limited to the above, and may include the brain, heart, lung field, liver, stomach, large intestine, or the like.

The following description describes an example where a diagnosis is made using a chest CT image obtained as a medical image.

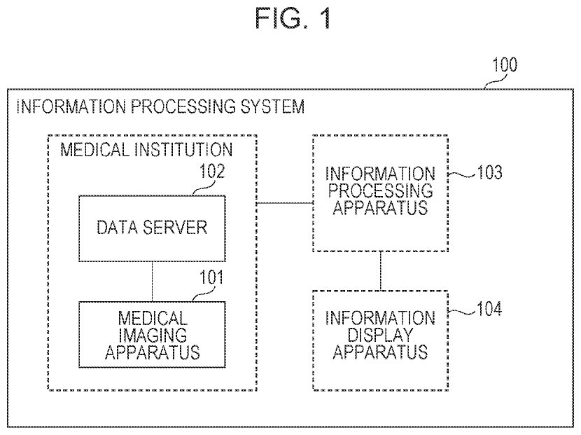

FIG. 1 is a diagram showing the overall configuration of an information processing system including an information processing apparatus according to the present embodiment.

The information processing system includes a medical imaging apparatus 101, a data server 102, an information processing apparatus 103, and an information display apparatus 104.

The medical imaging apparatus 101 is installed in a medical institution, such as a hospital, and captures an image of a subject to generate a medical image. Note that in the present embodiment, the image refers not only to an image displayed on a display unit, but also to an image stored as image data in a database or storage unit.

The data server 102 holds and manages, via a network, medical images of subjects captured by the medical imaging apparatus 101 and information associated with the medical images. For example, a medical image and the information associated with the medical image may be stored in a format that conforms to the Digital Imaging and Communication in Medicine (DICOM) standard which is an international standard that defines the format and communication procedure for medical images. However, if the medical image and the information about the medical image can be stored in association with each other, a standard other than the DICOM standard can be used. The associated information may also be stored in a file or database separate from the medical image.

In this case, the information processing apparatus 103 may access the file or database as necessary and refer to related information. The data server 102 may be an in-hospital system or an out-of-hospital system.

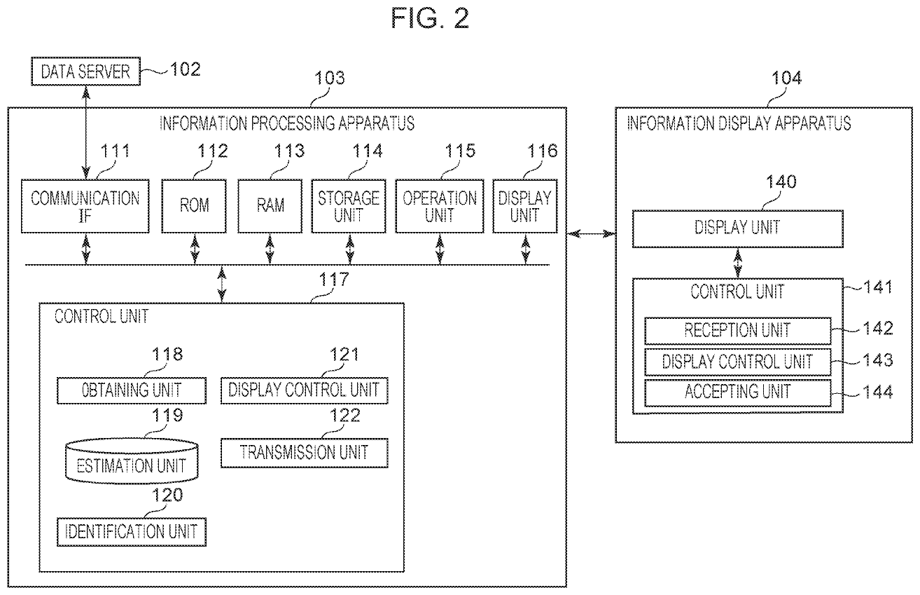

The information processing apparatus 103 can obtain medical images stored in the data server 102 via the network as shown in FIG. 2. The information processing apparatus 103 includes a communication IF (Interface) 111, a ROM (Read Only Memory) 112, a RAM (Random Access Memory) 113, a storage unit 114, an operation unit 115, a display unit 116, and a control unit 117.

The communication IF 111 is realized by a LAN card or the like, and controls communication between an external apparatus (for example, the data server 102) and the information processing apparatus 104. The ROM 112 is realized by a non-volatile memory or the like, and stores various programs and the like. The RAM 113 is realized by a volatile memory or the like, and temporarily stores various types of information. The storage unit 114 is an example of a computer-readable storage medium implemented by a large-capacity information storage apparatus typified by a hard disk drive (HDD) or a solid state drive (SSD), and stores various types of information. The operation unit 115 is realized by a keyboard, a mouse, or the like, and inputs an instruction from a user to the apparatus. The display unit 116 is an apparatus for displaying various types of information generated by the control unit 117. Typically, a liquid crystal display is used, but other types of displays, such as a plasma display, an organic EL display, a FED or the like may also be used.

More specifically, among the functional elements of the control unit 117, the display control unit 121 causes the display unit 116 to display various types of information. The control unit 117 is implemented by a CPU (Central Processing Unit), a GPU (Graphical Processing Unit), or the like, and performs overall control of each process in the information processing apparatus 104.

The control unit 117 includes, as its functional elements, an obtaining unit 118, an estimation unit 119, an identification unit 120, a display control unit 121, and a transmission unit 122.

The obtaining unit 118 reads and obtains from the data server 102 a medical image of a subject captured by the medical imaging apparatus 101 and information associated with the medical image. The information associated with the medical image may include subject information such as a subject ID, subject height, weight, age, gender, body fat, blood pressure, pregnancy status, heart rate, body temperature, etc. It may be examination information such as a condition, an imaging region, an imaging date and time, or an imaging location. The obtaining unit 118 may obtain all of the information associated with the medical images stored in the data server 102, or may obtain only some items. In a case where some items are obtained, the obtaining unit 118 may automatically obtain predetermined information or may also obtain information about items selected by a user via the operation unit 115. The data does not necessarily have to be obtained from the data server 102, and for example, data transmitted directly from the medical imaging apparatus 101 may be obtained. Moreover, the data server from which the data is obtained may differ depending on the information to be obtained. For example, the medical images and the information associated with the medical images may be obtained from different data servers.

The estimation unit 119 estimates a diagnosis result from the medical image of the subject obtained by the obtaining unit 118. In the present embodiment, a learning model that has performed deep learning in advance is used to estimate the diagnosis result from the medical image of the subject. The learning model, which will be described in detail later, is constructed, for example, by performing supervised learning with a neural network, using pairs of input data and labels as training data. In the present embodiment, the diagnosis result indicates, for example, the presence or absence of disease, the severity of disease (stage), the type of disease, the presence or absence of metastasis, the location of metastasis, the location of tumor, the size of tumor, or an identification result for an item such as the number of tumors. In the present embodiment, a configuration using a learning model for estimating the severity (disease stage) of a disease is described as an example, but a configuration for estimating any or all of the information can be used. The learning model can also perform iterative learning based on training data including input data and labels. The learning model may also be used to learn another model through transfer learning or fine-tuning, or further learning (additional learning) may be performed on the learning model. In the present embodiment, the learning model for estimating the diagnosis result may be generated by a learning unit (not shown) included in the information processing apparatus 103, or may be a model generated by an information processing apparatus other than the information processing apparatus 103. Furthermore, the specific algorithm for generating the learning model is not limited to the above, and in addition to deep learning using neural networks, for example, support vector machines, Bayesian networks, or random forests may be used.

The identification unit 120 determines a treatment method for the disease based on the diagnosis result estimated by the estimation unit 119.

The transmission unit 121 transmits the treatment method determined by the identification unit 120 to the information display apparatus 104.

The information display apparatus 104 is, for example, an apparatus that displays various types of information transmitted from the information processing apparatus 103, and the information display apparatus 104 typically uses a device such as a smartphone or a tablet terminal equipped with a liquid crystal display. Other types of displays such as a plasma display, an organic EL display, or FED may be used. It does not necessarily have to have a display, as long as it can display information. For example, it can be a device using AR (Augmented Reality) technology to display information in space.

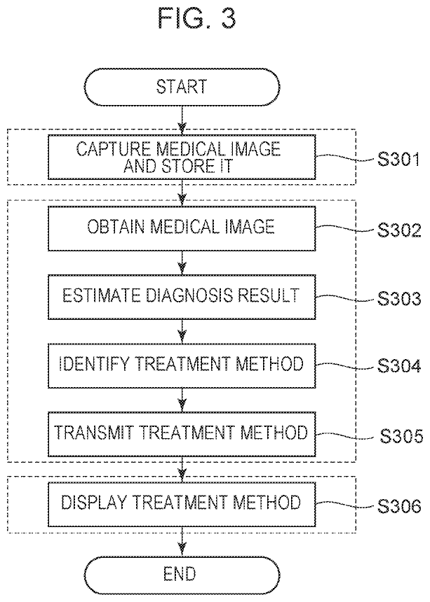

Next, a processing procedure performed in the information processing system 100 according to the present embodiment is described with reference to a flowchart shown in FIG. 3.

S301: Capturing/storing medical image

In S301, the medical imaging apparatus 101 captures a medical image of a subject, and stores it in the data server 102 via the network together with subject information or examination information.

S302: Obtaining medical image

In S302, the obtaining unit 118 included in the information processing apparatus 103 obtains the medical image captured in 5301 and information associated with the medical image from the data server 102.

S303: Estimating diagnosis result

In S303, the estimation unit 119 included in the information processing apparatus 103 estimates a diagnosis result by inputting the medical image of the subject obtained in S302 into the learning model.

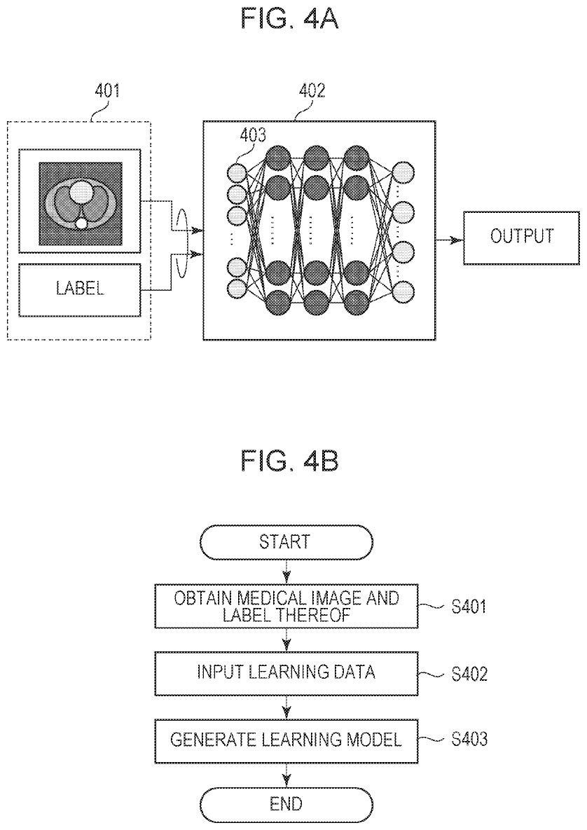

A method of generating a learning model used to estimate diagnosis results is described here with reference to FIGS. 4A and 4B.

In the present embodiment, the learning model is constructed by performing supervised learning using a neural network using pairs of medical images as input data and labels as output diagnosis results as training data. Although an example in which a learning model is generated by a learning unit (not shown) included in the control unit 117 is described below, the learning model may be generated by an information processing apparatus other than the information processing apparatus 103.

In S401, the learning unit included in the control unit 117 obtains medical images and labels that serve as training data from the data server 102. Note that the medical images and labels do not necessarily have to be obtained from the data server 102, and may be obtained from another data server. Here, in the present embodiment, the labels are identification information identifying, for example, the presence or absence of disease, the severity of the disease (stage), the type of disease, the presence or absence of metastasis, the location of the metastasis, the location of the tumor, the size of the tumor, the number of tumors, or the like.

In S402, the learning unit receives the set of the medical images and labels obtained in S401 as training data 401.

In S403, the learning unit generates a learning model by performing supervised learning using pairs of medical images and labels as the training data 401.

The learning unit provides the set of input data and labels included in the training data to a neural network 402 configured by combining perceptrons, and performs forward propagation such that the weighting for each perceptron in the neural network 402 is changed such that the output of the neural network 402 becomes equal to the label. For example, in the present embodiment, the forward propagation is performed such that the identification information output by the neural network is the same as the label identification information.

After performing the forward propagation in the manner described above, the learning unit adjusts the weighting values so as to reduce the error in the output of each perceptron by a method called back propagation. More specifically, the learning unit calculates the error between the output of the neural network 402 and the label, and modifies the weighting values so as to reduce the calculated error.

Here, the neural network 402 has a structure in which a large number of processing units 403 are arbitrarily connected. Examples of processing units 403 include processing units for a convolution operation, normalization processing such as batch normalization, or processing using activation functions such as ReLU, Sigmoid, and Softmax, each having a set of parameters to describe the processing. These may be combined into a structure called a convolutional neural network, in which three to several hundred processing units are arranged in a layer-to-layer configuration to form a convolutional layer, a pooling layer, a total coupling layer, and an output layer, and connected in a layer-to-layer fashion to perform processing sequentially.

For example, in the convolutional layer, a filter with predetermined parameters is applied to the input image data to perform feature extraction such as edge extraction. The predetermined parameters in this filter correspond to the weights of the neural network, which are learned by repeating the forward and back propagation described above.

The pooling layer blurs the image output from the convolutional layer to allow for object misalignment. This makes it possible to regard the object as the same object even if its position fluctuates. By combining these convolutional and pooling layers, feature values can be extracted from the image.

In the fully-connected layer, image data whose features have been extracted through the convolutional layer and the pooling layer are connected to one node, and a value obtained by the conversion using the activation function is output. Here, the activation function is a function that sets all output values less than 0 to 0, and is used to send only the outputs equal to or greater than a certain threshold to the output layer as meaningful information.

The output layer converts the outputs from the fully-connected layer into probabilities using, for example, a softmax function, which is a function for performing multi-class classification, and outputs identification information based on the obtained probabilities. Note that the convolutional neural network also repeats the forward propagation and back propagation so as to reduce the error between the output and the label.

Note that the learning unit may learn all the medical images obtained in S401, or may learn only some of the obtained medical images. Furthermore, the learning unit may use divided images obtained by dividing an obtained medical image into a plurality of regions as the input data, or may extract only a partial region of interest from the obtained medical image and use the extracted part as the input data.

Furthermore, the learning unit may combine a plurality of labels for one piece of input data to form training data. For example, for one medical image, the type of disease and the presence or absence of metastasis are associated as labels to form training data, and a learning model is generated that outputs the type of disease and the presence or absence of metastasis.

Alternatively, different learning models may be generated for each label associated with input data. For example, a first learning model is generated from training data in which the type of disease is labelled for each medical image, and a second learning model is generated from the training data in which the presence or absence of metastasis is labelled for each medical image.

Alternatively, a plurality of learning models may be generated by associating the same label with different input data. For example, in S401, CT images and MRI images are obtained. Then, a first learning model is generated from training data configured such that the severity (the disease stage) of a disease is labelled for each obtained CT image, and furthermore, a second learning model is generated from training data configured such that the severity (the disease stage) of the disease is labelled for each MRI image.

S304: Identification of treatment method

In S304, the identification unit 120 included in the information processing apparatus 103 identifies a treatment method based on the diagnosis result estimated in S303.

First, the identification unit 120 extracts past diagnosis results that are highly similar to the above estimation result. In addition to the estimation result, the degree of similarity with respect to at least one of the subject information and the imaging information may be calculated to extract the diagnosis result. For example, among the subject information, information on the gender and the pregnancy status is added to the similarity calculation items.

Here, the degree of similarity between the estimation result and the past diagnosis result is calculated such that the higher the degree of similarity between the estimation result and the past diagnosis result, the higher the value.

For example, the identification unit 120 calculates the degree of similarity based on the number of words that appear in common in both the estimation result and the past diagnosis result. Alternatively, the identification unit 120 may obtain feature vectors of the estimation result and the past diagnosis result from words appearing in the estimation result and the past diagnosis result and may calculate the distance between the feature vectors as the degree of similarity. The identification unit 120 may use any method to calculate the degree of similarity between the estimation result and the past diagnosis result. For example, the degree of similarity may be calculated between the medical image of the subject to be estimated in S303 and a past captured medical image stored in the data server 102. For example, past diagnosis results are classified into a plurality of classes, and in S303, it is estimated into which of the pre-classified classes the medical image of the subject is classified, and the past diagnosis result corresponding to the estimated class is extracted.

Each item used in the similarity calculation may be weighted. For example, in a case where it is desired to obtain information about a past subject of the same sex (female) who is pregnant at the time of the medical examination, the weight of a relevant item is increased.

A treatment method used in the extracted past diagnosis result is then identified.

Even for similar diagnosis results (for example, a breast cancer), the optimal procedure to select may differ depending on the individual's health status and the degree of disease progression, and thus the identification unit 120 may identify a plurality of treatment methods.

More specifically, in the present embodiment, treatment methods related to breast cancer, such as total mastectomy, breast-conserving surgery or preoperative medication, are identified. The above treatment methods are merely examples and the treatment methods are not limited to these examples, and other treatment methods may be identified depending on the estimated disease and other factors.

In addition, it is not necessary to identify a plurality of treatment methods. For example, one treatment method may be identified if the likely treatment is clearly determined by the severity of the disease. In this case, it is displayed that there are no other treatment methods to compare.

S305: Transmission of treatment method

In S305, the transmission unit 122 included in the information processing apparatus 103 transmits the treatment method identified in S304 and information related to the treatment method via the network in response to a request from the information display apparatus 104. As described in further detail later, the information about the treatment method is, for example, evaluation values for a plurality of indicators of the treatment method.

The transmission unit 122 may be configured to first transmit only items that are candidates for the treatment method, and then to transmit only information about the treatment method selected by a user from among the candidates for the treatment method displayed on the information display apparatus 104. Alternatively, the transmission unit 122 may be configured to transmit all information related to the identified treatment method in response to a request from the user.

S306: Indication of treatment method

In S306, the information display apparatus 104 displays the information related to the treatment method transmitted from the information processing apparatus 103.

More specifically, as shown in FIG. 2, the information display apparatus 104 includes a display unit 140, such as a liquid crystal display, which displays a user interface for receiving an instruction from a user, and a display control unit 143 which controls the display of information on the display unit 140. The display control unit 143 displays the information received by the reception unit 142 on the display unit 140 such that the user can obtain the information on the treatment method displayed on the display unit 140.

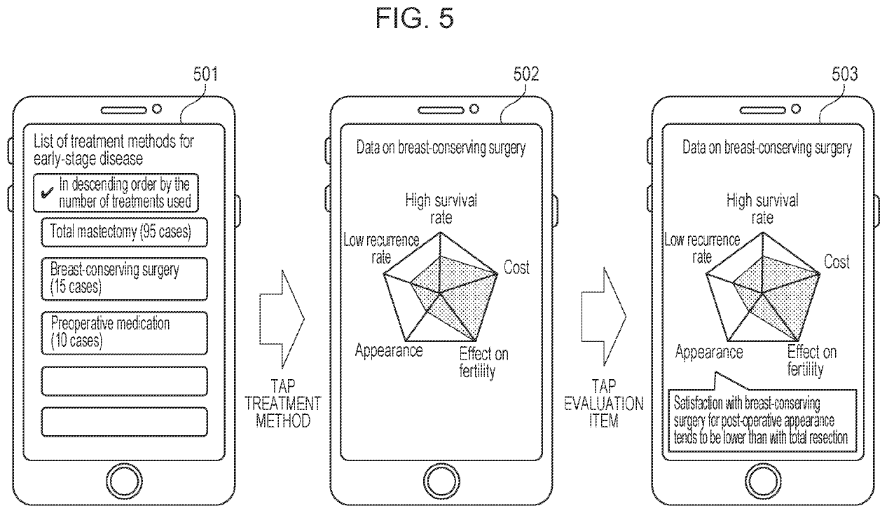

An example of a specific method of displaying information about the treatment method is described below with reference to FIG. 5.

A display screen 501 displays a list of information about treatment methods performed in past cases identified from diagnosis results estimated from a medical image of a subject. Here it is assumed by way of example that the display screen 501 displays information on a diagnosis result for a case of a subject diagnosed as breast cancer with a severity corresponding to an “early” stage and information is given relating to past cases diagnosed as “early”-stage breast cancer including cases (95 cases) in which total mastectomy was performed, cases (15 cases) in which breast-conserving surgery was performed, and cases (10 cases) in which preoperative medication was performed.

The above-described manner of displaying information on the display screen 501 is merely an example and is not limited thereto. For example, instead of displaying a list, the display control unit 143 may sequentially display candidate treatment methods that are most likely to be options for the user. The display control unit 143 may display the candidate treatment methods in descending order of the number of adopted treatment method in cases, as with the display screen 501, or in ascending order. Alternatively, the display control unit 143 may display the candidate treatment methods in the order from the standard treatment. Furthermore, the display control unit 143 may highlight the treatment methods that are frequently used as the treatment method. The control unit 141 may further have a search function of screening the extracted past cases based on the subject information and/or the like.

The accepting unit 144 included in the information display apparatus 104 accepts an arbitrary selection from the user regarding the treatment methods displayed on the display screen 501, and changes the display screen according to the selection.

A display screen 502 shows an example of what is displayed when a breast-conserving surgery is selected on the display screen 501. More specifically, on the display screen 502, evaluation values for indicators such as a high survival rate, a cost, an effect on fertility, an effect on appearance, a low recurrence rate are displayed in the form of a radar chart for a case in which the breast-conserving surgery is selected. A radar chart is also displayed when another treatment method displayed on the display screen 501 is selected, and the user can compare the evaluation values shown on these radar charts between the treatment methods. Note that the form of presentation of the evaluation values does not have to be a radar chart, but may be displayed in the form of various graphs and tables.

Various statistical values (average, median, maximum, etc.) based on the results evaluated in past cases may be used as evaluation values for the respective indicators. Different statistical values may be used for each indicator or the same statistical value may be used for all indicators.

There is a possibility that information on all indicators are not stored in association with past cases. Therefore, for example, the number of cases used to calculate the evaluation values may be displayed together with the evaluation values of the indicators.

Alternatively, in a case where the number of samples used to calculate the evaluation values is small, the fact that the reliability of the evaluation value is relatively low may be emphasized in the presentation.

When one of the displayed items is selected, supplementary information for the selected item is displayed as shown on a display screen 503. In this example, the display screen 503 displays information including supplemental information that is additionally displayed when “appearance” is selected from the items. This supplemental information indicates satisfaction with postoperative appearance compared to satisfaction with other treatment methods. Note that the supplemental information does not have to be information on the comparison with other treatment methods for each indicator. For example, the information may be about experiences by people who have chosen the treatment method displayed in the past, or the information may indicate questionnaire results etc. about the degree of satisfaction.

In this way, by comparing the evaluation values of the respective indicators represented on the chart and the supplementary information between the treatment methods, the user can consider the treatment method that is suitable for him/her based on objective information.

The indicators displayed on the display screen 502 may not necessarily be the five indicators described above, and may further include other indicators such as the level of side effect/complication risk, or the number of indicators may be less than five. Alternatively, a user's selection of an indicator may be accepted and an evaluation value for the selected indicator may be displayed, or an evaluation value for a predetermined indicator may be displayed regardless of the user's selection.

The process performed by the information processing system 100 has been described above.

According to the above, the user can make a comparison among a plurality of treatment methods and the evaluation values of the treatment methods. Even in a case where only one treatment method is suggested, the user can consider the treatment method based on the information of past cases. This allows the user to assess the appropriateness of the treatment method proposed in the diagnosis with the doctor and to understand the treatment that is appropriate for the user.

First Modification

In the embodiment described above, in S302, the obtaining unit 118 included in the information processing apparatus 103 obtains the medical image of the subject and information associated with the medical image from the data server 102.

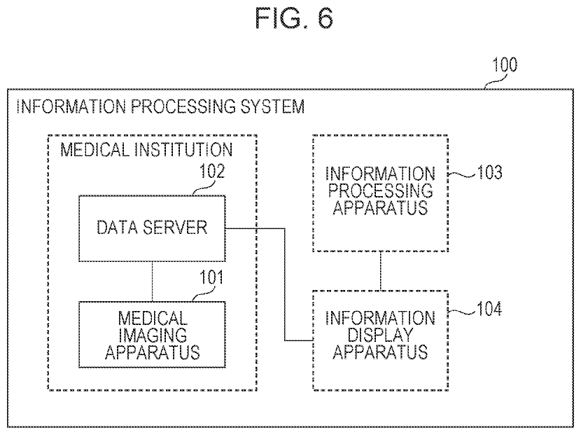

Alternatively, as shown in FIG. 6, in a case where the information display apparatus 104 can access the data server 102 that stores and manages medical images of subjects or subject information, the obtaining unit 118 of the information processing apparatus 103 may obtain each piece of information transmitted from the information display apparatus 104.

In this case, the user of the information display apparatus 104 can obtain information about treatment methods without having to go to a medical facility, thereby reducing restrictions of location and time.

Second Modification

The embodiment has been described above by way of example for a case where in S303, the estimation unit 119 included in the information processing apparatus 103 estimates a diagnosis result (more specifically, the severity of the disease) from a CT image of the chest of a subject.

However, in S303, the estimation unit 119 may correct the estimated diagnosis result of the subject using information of the subject or the result of a detailed examination using a microscope and/or the like. For example, by examining cells from a subject under a microscope, it is possible to obtain detailed information about the tumor size, the presence or absence of lymph node metastasis, the presence or absence of lymph vessel invasion, the presence or absence of venous invasion, the tumor type, the cell proliferation ability, the malignancy, the presence or absence of hormone receptors, the level of HER2 protein expression, and/or the like. Therefore, the estimation unit 119 may correct the result estimated from the medical image of the subject based on the result of the detailed examination. This makes it possible to estimate the diagnosis result of the subject more accurately.

In the detailed examination, for example, the diagnosis result is estimated by inputting the image of the subject captured using a microscope into the learning model in the same manner as described above with reference to S303.

That is, the diagnosis result estimated by inputting the medical image obtained by the first imaging apparatus into the first learning model may be corrected using the output result obtained by inputting the medical image obtained by the second imaging apparatus into the second learning model.

Third Modification

In the embodiment described above, in S306, the display control unit 143 included in the information display apparatus 104 displays the evaluation values of the indicators of each treatment method in a radar chart such that the user can compare the evaluation values corresponding to the respective treatment methods to be selected.

Alternatively, instead of switching the displayed treatment methods, the display control unit 143 may display the evaluation values of the indicators of the respective treatment methods in a superimposed or parallel manner. For example, on the display screen 501, the accepting unit 144 accepts a selection of a plurality of treatment methods from the user, and the display control unit 143 displays the evaluation values of the indicators of the plurality of selected treatment methods in a radar chart in a superimposed manner.

This allows the user to compare different treatment methods without having to change screens, which improves visibility.

### Other Embodiments

The present disclosure may be realized by supplying a program for realizing one or more functions of the one or more embodiments described above to a system or an apparatus via a network or a storage medium, and reading and executing the program by one or more processors of a computer in the system or the apparatus. The present disclosure may also be implemented by a circuit that realizes one or more functions.

The processor or the circuit may include a central processing unit (CPU), a microprocessing unit (MPU), a graphics processing unit (GPU), an application specific integrated circuit (ASIC), a field programmable gate array (FPGA). Furthermore, the processor or the circuit may include a digital signal processor (DSP), a data flow processor (DFP), or neural processing unit (NPU).

The information processing apparatus according to one of the above-described embodiments may be realized as a single apparatus, or may be realized in a form in which a plurality of apparatuses are communicatively combined to execute the above-described processing. It should be noted Note that any of these modifications falls within the scope of the embodiments of the invention. The processing described above may be executed by a common single server apparatus or by a group of servers. The information processing apparatus and the plurality of apparatuses constituting the information processing system need only be capable of communicating at a predetermined communication rate, and do not need to be located in the same facility or in the same country.

Embodiments of the present invention include an embodiment in which a software program to realize a function of the above-described embodiments is supplied to a system or apparatus, and a computer of the system or the apparatus reads and executes the supplied program.

That is, the program code itself installed in the computer to realize processing according to any embodiment also falls within the scope of the embodiments of the present invention. Furthermore, a function of the embodiments can also be realized by an OS or the like running on a computer by performing part or all of actual processes according to an instruction included in a program read by the computer.

The present invention is not limited to the embodiments described above, but various changes and modifications are possible without departing from the spirit and the scope of the present disclosure. Therefore, the following claims are appended in order to make public the scope of the present invention.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

1. An information processing apparatus comprising:
an estimation unit configured to estimate a diagnosis result for a medical image of a subject by using a learning model learned by a set of a medical image and a diagnosis result of the medical image; and
an output unit configured to output a candidate for a treatment method to be applied to the estimated diagnosis result and an evaluation value for the candidate for the treatment method.

2. The information processing apparatus according to claim 1, further comprising an identification unit configured to identify a treatment method performed in a past case extracted based on a degree of similarity with the estimated diagnosis result,
wherein the output unit outputs the treatment method identified by the identification unit as a candidate treatment method to be applied to the diagnosis result.

3. The information processing apparatus according to claim 1, wherein in a case where there are two or more candidates for the treatment method, the output unit outputs at least two candidates for the treatment method, while in a case where there is only one candidate for the treatment method, the output unit outputs the only one candidate for the treatment method together with information indicating that there are no other candidates for the treatment method.

4. The information processing apparatus according to claim 1, wherein the diagnosis result is an identification result for at least one of following:
presence or absence of a disease; a severity of the disease;
a type of the disease; presence or absence of metastasis; a location of the metastasis; a location of a tumor; a size of the tumor; and a number of tumors.

5. The information processing apparatus according to claim 1, wherein the learning model is constructed so as to include a neural network that performs deep learning on training data including a set of a medical image and a diagnosis result of the medical image.

6. The information processing apparatus according to claim 1, further comprising a correction unit configured to correct the diagnosis result estimated by the estimation unit by inputting the medical image of the subject captured by a first imaging apparatus into a first learning model, using an output result obtained by inputting a medical image captured by a second imaging apparatus into a second learning model.

7. The information processing apparatus according to claim 1, further comprising a correction unit configured to correct the diagnosis result estimated by the estimation unit based on at least one of following information: a diagnosis result for a medical image captured by an imaging apparatus different from the imaging apparatus by which the medical image is captured; subject information of the subject; and an imaging condition under which the image of the subject is captured.

8. An information display apparatus comprising:
an obtaining unit configured to obtain a candidate for a treatment method to be applied to a diagnosis result estimated from a medical image of a subject and an evaluation value for the candidate for the treatment method; and
a display control unit configured to display on a display unit the candidate for the treatment method and the evaluation value for the candidate for the treatment method obtained by the obtaining unit.

9. The information display apparatus according to claim 8, wherein
the obtaining unit obtains, as candidates for the treatment method to be applied to the diagnosis result, a first treatment method and an evaluation value for the first treatment method, and a second treatment method and an evaluation for the second treatment method, and
the display control unit displays the evaluation value for the first treatment method and the evaluation value for the second treatment method on a display unit in a parallel manner, a superimposed manner, a superimposed manner, or a switchable manner.

10. The information display apparatus according to claim 8, wherein
the obtaining unit obtains a first treatment method, an evaluation value for the first treatment method, and information indicating that there are no other candidates for the treatment method; and
the display control unit displays the evaluation value for the first treatment method and the information indicating that there are no other candidates for the treatment method on the display unit.

11. The information display apparatus according to claim 8, wherein the display control unit further displays supplementary information related to the evaluation value.

12. An information processing system comprising: an information processing apparatus comprising
an estimation unit configured to estimate a diagnosis result for a medical image of a subject by using a learning model learned by a set of a medical image and a diagnosis result of the medical image; and
an output unit configured to output a candidate for a treatment method to be applied to the estimated diagnosis result and an evaluation value for the candidate for the treatment method;
and an information display apparatus comprising:
an obtaining unit configured to obtain, from an output provided by the information processing apparatus, a candidate for a treatment method to be applied to a diagnosis result estimated from a medical image of a subject and an evaluation value for the candidate for the treatment method; and
a display control unit configured to display on a display unit the candidate for the treatment method and the evaluation value for the candidate for the treatment method obtained by the obtaining unit.

13. The information processing system according to claim 12, wherein
the information display apparatus further comprises a transmission unit configured to transmit a medical image captured for the subject to the information processing apparatus, and
the estimation unit included in the information processing apparatus estimates a diagnosis result for the medical image of the subject transmitted from the information display apparatus.

14. An information processing system comprising:
an estimation unit configured to estimate a diagnosis result regarding a breast disease from a medical image of a chest of a subject using a learning model learned using a set of a medical image and a diagnosis result for the medical image;
an identification unit configured to identify a treatment method for the breast disease performed in a past case extracted based on the degree of similarity with the estimated diagnosis result; and
a display control unit configured to display on a display unit the candidate for the treatment method and the evaluation value for the candidate for the treatment method identified by the identification unit.

15. The information processing system according to claim 14, wherein the display control unit displays on a display unit an evaluation value for at least one of following indicators: a survival rate; a cost; a side effect; an effect on appearance; an effect on fertility; and
a low recurrence rate.

16. An information processing method comprising:
estimating a diagnosis result for a medical image of a subject by using a learning model learned by a set of a medical image and a diagnosis result of the medical image;
identifying a treatment method performed in a past case extracted based on a degree of similarity with the estimated diagnosis result; and
displaying on a display unit a candidate for the treatment method and an evaluation value for the candidate for the treatment method identified by the identification unit.

17. A non-transitory computer-readable storage medium storing a program for causing a computer to execute the method according to claim 16.
